(19) United States
(12) Patent Application Publication
(10) Pub. No.: US 2025/0366724 A1
(43) Pub. Date: Dec. 4, 2025

(54) BLOOD PRESSURE MEASURING DEVICE

(71) Applicant: Quanta Computer Inc., Taoyuan City (TW)

(72) Inventors: Jung-Wen CHANG, Taoyuan City (TW); Chien-Hung LIN, Taoyuan City (TW)

(21) Appl. No.: 18/793,299

(22) Filed: Aug. 2, 2024

(30) Foreign Application Priority Data
Jun. 3, 2024 (TW) 113120453

(51) Int. Cl. A61B 5/021; A61B 5/00; A61B 5/0205; A61B 5/11; A61B 5/28

(52) U.S. Cl. CPC A61B 5/02125; A61B 5/02141; A61B 5/1102; A61B 5/28; A61B 5/6824; A61B 5/6825; A61B 5/7271; A61B 5/0205; A61B 2560/0468; A61B 2562/166; A61B 2562/227

(57) ABSTRACT

A blood pressure measuring device is provided. The blood pressure measuring device includes a first measuring assembly, a second measuring assembly, a first wearable device, and a second wearable device. The first measuring assembly includes a plurality of sensing elements that are at least partially in contact with human skin. The second measuring assembly includes a sensing element that is in contact with human skin. The first measuring assembly is installed on the first wearable device. The second measuring assembly is installed on the second wearable device.

### CROSS REFERENCE TO RELATED APPLICATIONS

This application claims priority of Taiwan Patent Application No. 113120453, filed on Jun. 3, 2024, the entirety of which is incorporated by reference herein.

### BACKGROUND OF THE INVENTION

### Field of the Invention

The present invention relates to a blood pressure measuring device, and, in particular, to a blood pressure measuring device that does not require a cuff.

### Description of the Related Art

Traditional sphygmomanometers, including mercury sphygmomanometers and electronic sphygmomanometers, require inflatable cuffs to measure blood pressure. They not only consume much time and work, but also have the problem of being bulky and difficult to carry. This makes it difficult for users to monitor their health status anytime and anywhere.

In addition, devices that use ECG signals to measure blood pressure are known. They require both hands to press in order to form a circuit loop between blood vessels and the heart. This reduces the convenience of use.

Accordingly, how to measure blood pressure without using a cuff or using both hands has become an important issue.

### BRIEF SUMMARY OF THE INVENTION

An embodiment of the present invention provides a blood pressure measuring device. The blood pressure measuring device includes a first measuring assembly, a second measuring assembly, a first wearable device, and a second wearable device. The first measuring assembly includes a plurality of sensing elements that are at least partially in contact with human skin. The second measuring assembly includes a sensing element that is in contact with human skin. The first measuring assembly is installed on the first wearable device. The second measuring assembly is installed on the second wearable device.

In addition, an embodiment of the present invention provides a blood pressure measuring device, including a measuring assembly, a wearable device, and a calculating element. The measuring assembly includes a plurality of sensing elements that are at least partially in contact with human skin. The measuring assembly is installed on the wearable device. The blood pressure measuring device does not include an inflatable cuff. The sensing elements of the measuring assembly include a PPG sensor and a BCG sensor, and a PPG signal is obtained from the PPG sensor, while a BCG signal is obtained from the BCG sensor. The calculating element calculates a time difference between the PPG signal and the BCG signal, and estimates a blood pressure from the time difference.

### BRIEF DESCRIPTION OF THE DRAWINGS

Aspects of the present disclosure are best understood from the following detailed description when read with the accompanying figures. It should be noted that, in accordance with the standard practice in the industry, various features are not drawn to scale. In fact, the dimensions of the various features may be arbitrarily increased or reduced for clarity of discussion.

FIG. 1 shows a schematic view of a blood pressure measuring device that is worn on human body, according to some embodiments of the present disclosure;

FIG. 2A shows a perspective schematic view of the blood pressure measuring device, according to the first embodiment of the present disclosure;

FIG. 2B shows the internal configuration and appearance of the first wearable device, according to the first embodiment of the present disclosure;

FIG. 3A shows a perspective schematic view of the blood pressure measuring device, according to the second embodiment of the present disclosure;

FIG. 3B shows the internal configuration and appearance of the first wearable device, according to the second embodiment of the present disclosure;

FIG. 4A shows a perspective schematic view of the blood pressure measuring device, according to the third embodiment of the present disclosure;

FIG. 4B shows the internal configuration and appearance of the first wearable device, according to the third embodiment of the present disclosure;

FIG. 5A shows a perspective schematic view of the blood pressure measuring device, according to the fourth embodiment of the present disclosure;

FIG. 5B shows the internal configuration and appearance of the first wearable device, according to the fourth embodiment of the present disclosure;

FIGS. 6A and 6B show waveforms of the ECG signal, the BCG signal, and the PPG signal, according to some embodiments of the present disclosure;

FIG. 7A shows the correspondence between the time differences and blood pressures, according to some embodiments of the present disclosure;

FIG. 7B shows the calibration curve formed based on the correspondence in FIG. 7A, according to some embodiments of the present disclosure; and

FIG. 7C shows the calibration curve formed based on the correspondence in FIG. 7A, according to some embodiments of the present disclosure.

### DETAILED DESCRIPTION OF THE INVENTION

The following description is made for the purpose of illustrating the general principles of the invention and should not be taken in a limiting sense. The scope of the invention is best determined by reference to the appended claims.

The following disclosure provides many different embodiments, or examples, for implementing different features of the provided subject matter. Specific examples of components and arrangements are described below to simplify the present disclosure. These are, of course, merely examples and are not intended to be limiting. For example, the formation of a first feature over or on a second feature in the description that follows may include embodiments in which the first and second features are formed in direct contact, and may also include embodiments in which additional features may be formed between the first and second features, such that the first and second features may not be in direct contact.

In addition, the present disclosure may repeat reference numerals and/or letters in the various embodiments. This repetition is for the purpose of simplicity and clarity and does not in itself dictate a relationship between the various embodiments and/or configurations discussed. Moreover, the formation of a feature on, connected to, and/or coupled to another feature in the present disclosure that follows may include embodiments in which the features are formed in direct contact, and may also include embodiments in which additional features may be formed interposing the features, such that the features may not be in direct contact. In addition, spatially relative terms, for example, “vertical,” “above,” “over,” “below,”, “bottom,” etc. as well as derivatives thereof (e.g., “downwardly,” “upwardly,” etc.) are used for ease of the present disclosure of one features relationship to another feature. The spatially relative terms are intended to cover different orientations of the device including the features.

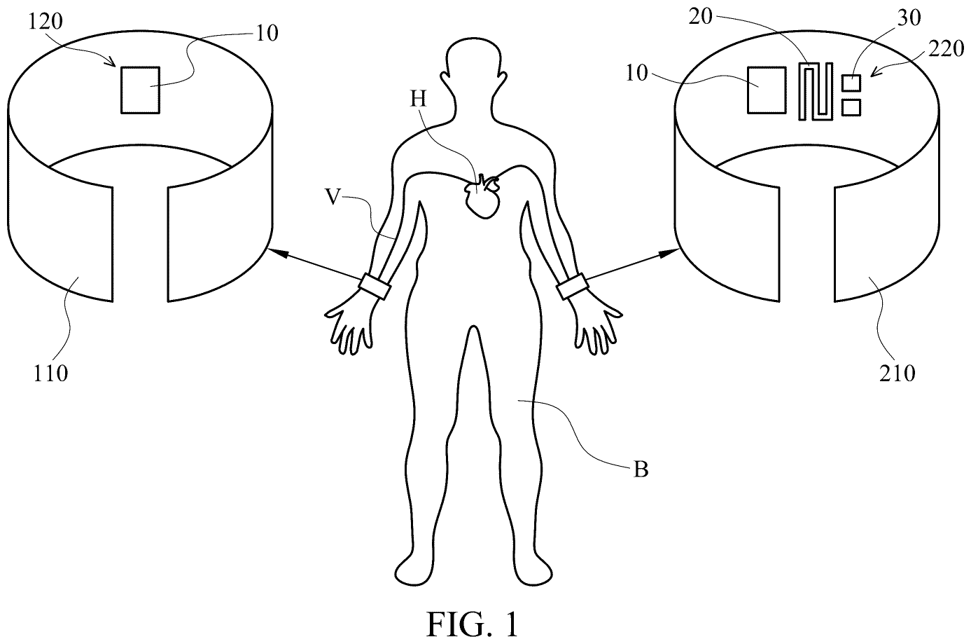

FIG. 1 shows a schematic view of the blood pressure measuring device that is worn on human body B, according to some embodiments of the present disclosure. The blood pressure measuring device includes a first wearable device 110 and a second wearable device 210. In the embodiment shown in FIG. 1, the first wearable device 110 and the second wearable device 210 are respectively worn on the wrist parts of two hands on the human body B. The first wearable device 110 and the second wearable device 210 may be in the form of wristbands, but they are not limited thereto. For example, the first wearable device 110 and the second wearable device 210 may also be watches that are worn on the wrist parts as well. Or, the first wearable device 110 and the second wearable device 210 may be rings that are worn on fingers. The first wearable device 110 and the second wearable device 210 that are worn on both hands of human body B can contact blood vessels V by touching the surface of the skin to form a circuit loop that is connected to the heart H to measure the required signals. The way of measuring signals will be described in details below.

The blood pressure measuring device includes a first measuring assembly 120 on the first wearable device 110, and includes a second measuring assembly 220 on the second wearable device 210. In FIG. 1, the first measuring assembly 120 includes an ECG sensor 10, and the second measuring assembly 220 includes an ECG sensor 10, a BCG sensor 20, and a PPG sensor 30. However, the left and right configuration of the sensors is not limited to the embodiment shown in FIG. 1. For example, although in FIG. 1, the first wearable device 110 is worn on the right hand of human body B and the second wearable device 210 is worn on the left hand of human body B, in other embodiments, the first wearable device 110 may be worn on the left hand of human body B and the second wearable device 210 may be worn on the right hand of human body B. The first wearable devices 110 and second wearable devices 210 shown in embodiments below do not have restrictions on left or right configurations. They may be swapped according to actual needs.

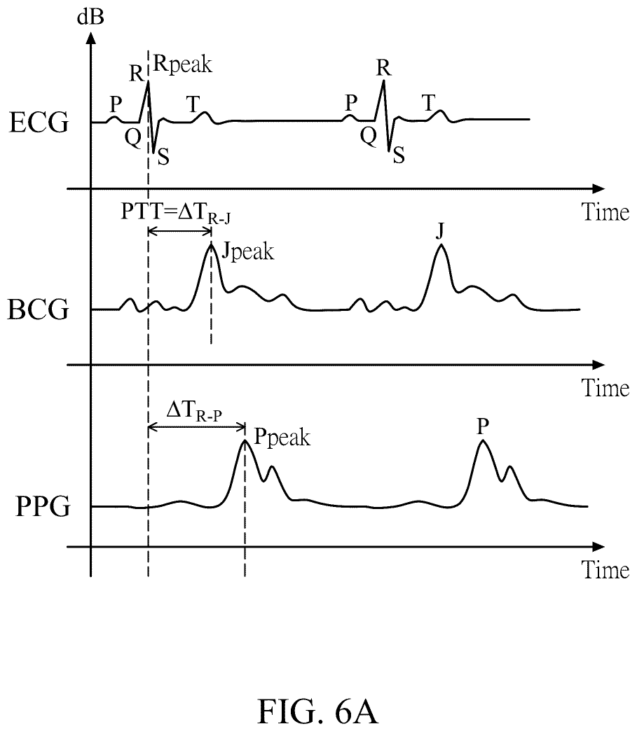

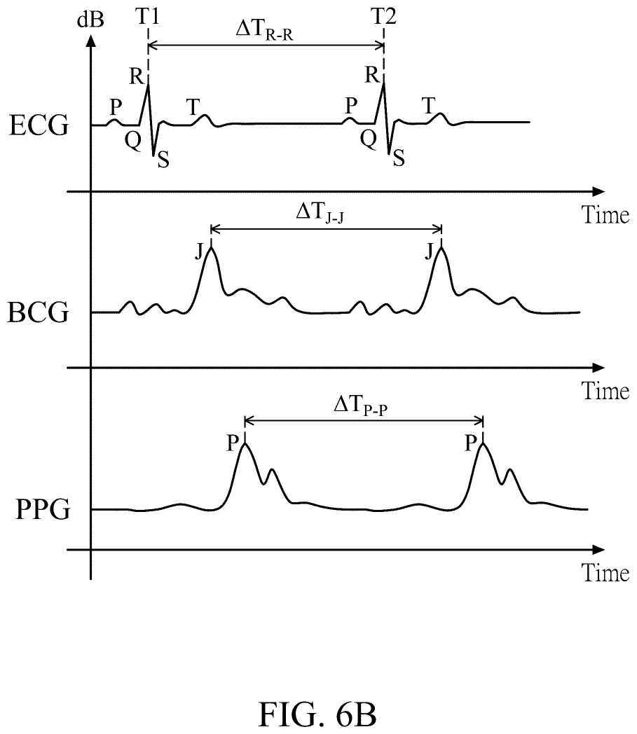

Here, the ECG (electrocardiography) sensor 10 is a type of sensor that measures electrocardiograms. Electrodes are disposed inside the ECG sensor 10. They capture electrical signals by touching human skin. When in use, the electrodes of the ECG sensor 10 need to be positioned on either side of the heart H so that voltage changes between the two electrodes are recorded, so as to show or indicate the rhythm of the heartbeat. In some embodiments according to the present disclosure, an ECG signal, i.e., an electrical signal generated by the heart, is obtained by the ECG sensor 10 of the first measuring assembly 120 and the ECG sensor 10 of the second measuring assembly 220. As shown in the waveforms in FIGS. 6A and 6B, the R-peak of the ECG signal represents ventricular contraction. The time difference ΔT_{R-R} between two R-peaks is regarded as the time for the heart to beat once.

Here, the BCG (ballistocardiography) sensor 20 is a sensor for measuring the pressure signal of force changes caused by the beating of the heart. The BCG sensor 20 may include a fiber optic sensing structure, in which the fiber optic material is deformed when subjected to a slight external force or shock of vibration, resulting in a change in the intensity of the light, thereby registering or recording the pressure signal to indicate a heart shock wave. When in use, the BCG sensor 20 only needs to be attached to the body surface, regardless of whether it is in direct contact with the skin of the human body B, as long as it can detect or measure the vibration of the skin. In some embodiments according to the present disclosure, a BCG signal, i.e., heart shock wave signal (pressure signal), is obtained by the BCG sensor 20. As shown in the waveforms of FIGS. 6A and 6B, the J-peak of the BCG signal represents vasoconstriction. The time difference ΔT_{J-J} between two J-peaks is regarded as the time for the heart to beat once.

Here, the PPG (photoplethysmography) sensor 30 is a sensor that measures light signals from changes in blood vessel volume. The PPG sensor 30 may include a light sensing element that detects changes of light in blood vessels subjected to blood flow. Different light signals are generated during systole and diastole to indicate the rhythm of the heartbeat. In some embodiments according to the present disclosure, a PPG signal, i.e., a vascular volume change signal (optical signal), is obtained by the PPG sensor 30. As shown in the waveforms of FIGS. 6A and 6B, the P-peak of the PPG signal represents the contraction of the heart, and the time difference ΔT_{P-P} between two P-peaks is regarded as the time for the heart to beat once.

In theory, the time difference ΔT_{R-R} between two R-peaks, the time difference ΔT_{J-J} between two J-peaks, and the time difference ΔT_{P-P} between two P-peaks should be approximately equal.

Vascular pressure is related to the time of heart pulse transmission. The higher the blood pressure, the faster the heart pulse travels. Similarly, the lower the blood pressure, the slower the heart pulse is transmitted. Therefore, the blood pressure of the test subject can be estimated by calculating the time difference between any two of the following peaks: the R-peak of the ECG signal, the J-peak of the BCG signal, and the P-peak of the PPG signal.

As shown in FIG. 6A, in the sequence of the timeline, the R-peak of the ECG signal occurs earliest, followed by the J-peak of the BCG signal, and finally the P-peak of the PPG signal. Moreover, the J-peak and the P-peak both occur between two R-peaks.

When using the blood pressure measurement device according to some embodiments of the present disclosure, the measurement functions of the ECG sensor 10, the BCG sensor 20, and the PPG sensor 30 are first activated to measure the wearer's ECG signal, BCG signal, and PPG signal, respectively. The sampling times of the three signals are then synchronized (the timelines shown in FIG. 6A are the timelines after synchronization) and the values are normalized for calculation. Next, the time point at which the R-peak of the ECG signal occurs, the time point at which the J-peak of the BCG signal occurs, and the time point at which the P-peak of the PPG signal occurs are recorded, and the average time difference between any two of the three peaks is calculated. Finally, a blood pressure value is estimated from this time difference. The aforementioned time difference is also called pulse transit time (PTT).

It was mentioned above that the average time difference is calculated by selecting any two of the three peaks, where the selected signals may vary depending on the implementation. Various embodiments according to the present disclosure will be described below with reference to the drawings.

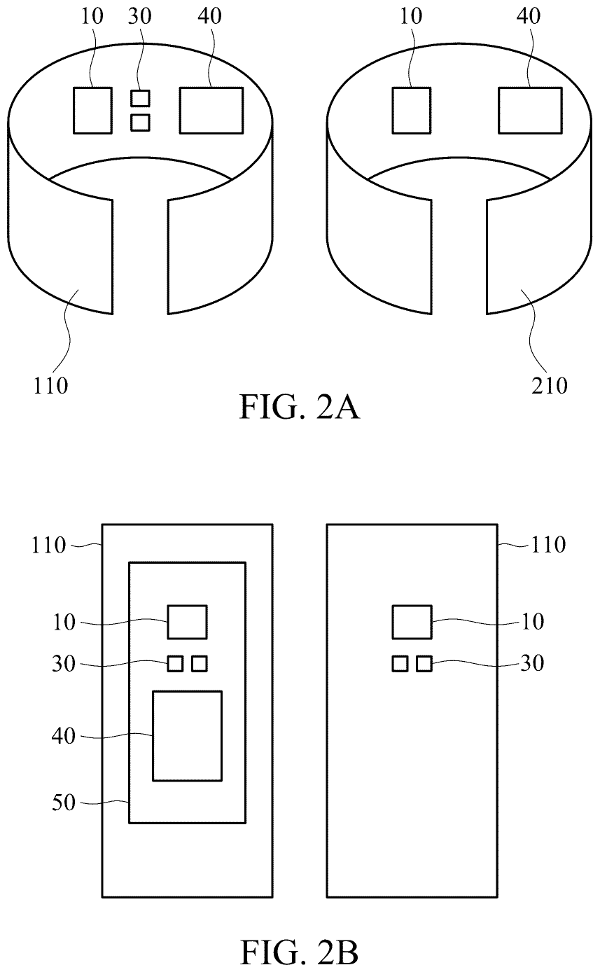

FIG. 2A shows a perspective schematic view of the blood pressure measuring device, according to the first embodiment of the present disclosure. FIG. 2B shows the internal configuration and appearance of the first wearable device 110, according to the first embodiment of the present disclosure.

In the first embodiment, the first measuring assembly 120 on the first wearable device 110 includes an ECG sensor 10, a PPG sensor 30, and a power supply element 40. The second measuring assembly 220 on the second wearable device 210 includes an ECG sensor 10 and a power supply element 40. The power supply element 40 may be a battery or any suitable power supply elements. As shown in FIG. 2B, the ECG sensor 10, the PPG sensor 30 and the power supply element 40 are disposed on the flexible printed circuit board 50 and electrically connected to the flexible printed circuit board 50. The flexible printed circuit board 50 is embedded inside the first wearable device 110. When viewed from outside, as shown on the right side of FIG. 2B, only the ECG sensor 10 and the PPG sensor 30 are exposed from the first wearable device 110 in order to make contact with the human body B.

In the first embodiment, the time difference ΔT_{R-P} between the R-peak of the ECG signal and the P-peak of the PPG signal may be measured, as shown in FIG. 6A. The wearer's blood pressure can be estimated from the time difference ΔT_{R-P}. The estimation method will be described in details below.

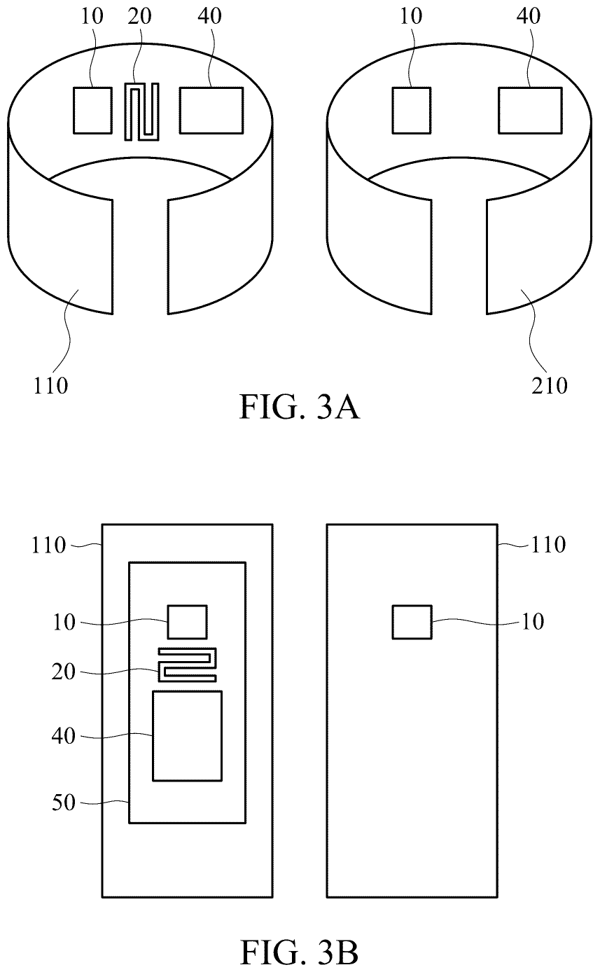

FIG. 3A shows a perspective schematic view of the blood pressure measuring device, according to the second embodiment of the present disclosure. FIG. 3B shows the internal configuration and appearance of the first wearable device 110, according to the second embodiment of the present disclosure.

In the second embodiment, the first measuring assembly 120 on the first wearable device 110 includes an ECG sensor 10, a BCG sensor 20, and a power supply element 40. The second wearable device 210, which is similar to that in the first embodiment, only includes an ECG sensor 10 and a power supply element 40. As shown in FIG. 3B, the ECG sensor 10, the BCG sensor 20 and the power supply element 40 are disposed on the flexible printed circuit board 50 and electrically connected to the flexible printed circuit board 50. The flexible printed circuit board 50 is embedded inside the first wearable device 110. When viewed from outside, as shown on the right side of FIG. 3B, only the ECG sensor 10 is exposed from the first wearable device 110 in order to make contact with the human body B. As mentioned above, the BCG sensor 20 only needs to sense vibrations. Thus, it does not need to be in direct contact with the skin of human body B.

In the second embodiment, the time difference ΔT_{R-J} between the R-peak of the ECG signal and the J-peak of the BCG signal may be measured, as shown in FIG. 6A. The wearer's blood pressure can be estimated from the time difference ΔT_{R-J}. The estimation method will be described in details below.

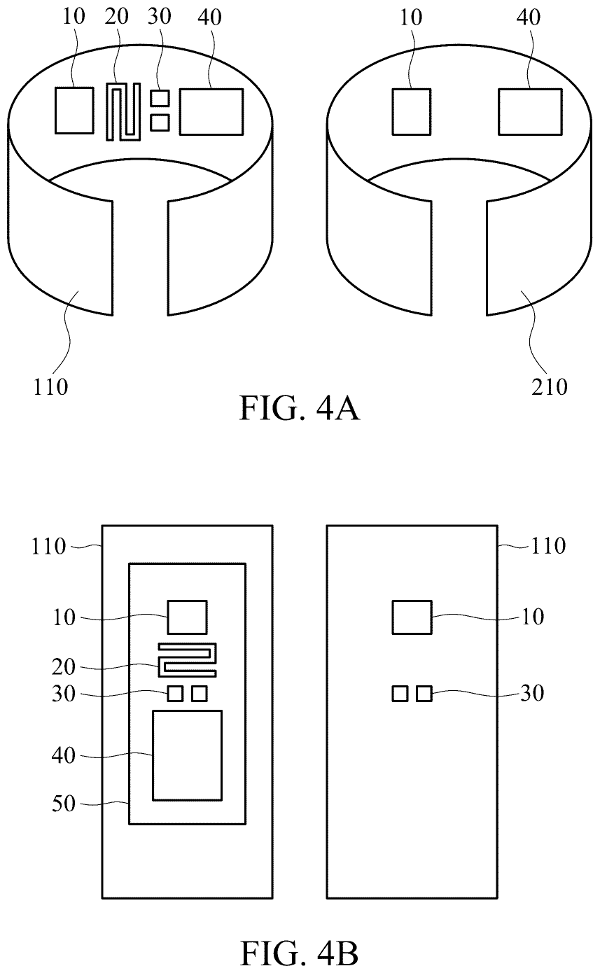

FIG. 4A shows a perspective schematic view of the blood pressure measuring device, according to the third embodiment of the present disclosure. FIG. 4B shows the internal configuration and appearance of the first wearable device 110, according to the third embodiment of the present disclosure.

In the third embodiment, the first measuring assembly 120 on the first wearable device 110 includes an ECG sensor 10, a BCG sensor 20, a PPG sensor 30, and a power supply element 40. The second wearable device 210, which is similar to that in the first embodiment, only includes an ECG sensor 10 and a power supply element 40. As shown in FIG. 4B, the ECG sensor 10, the BCG sensor 20, the PPG sensor 30, and the power supply element 40 are disposed on the flexible printed circuit board 50 and electrically connected to the flexible printed circuit board 50. The flexible printed circuit board 50 is embedded inside the first wearable device 110. When viewed from outside, as shown on the right side of FIG. 4B, only the ECG sensor 10 and the PPG sensor 30 are exposed from the first wearable device 110 to be in contact with the human body B. As mentioned above, the BCG sensor 20 only needs to sense vibrations. Thus, it does not need to be in direct contact with the skin of human body B.

In the third embodiment, the time difference ΔT_{R-J} between the R-peak of the ECG signal and the J-peak of the BCG signal and the time difference ΔT_{R-P} between the R-peak of the ECG signal and the P-peak of the PPG signal may be measured, as shown in FIG. 6A. The wearer's blood pressure can be estimated from the time difference ΔT_{R-J} and the time difference ΔT_{R-P}. The estimation method will be described in details below.

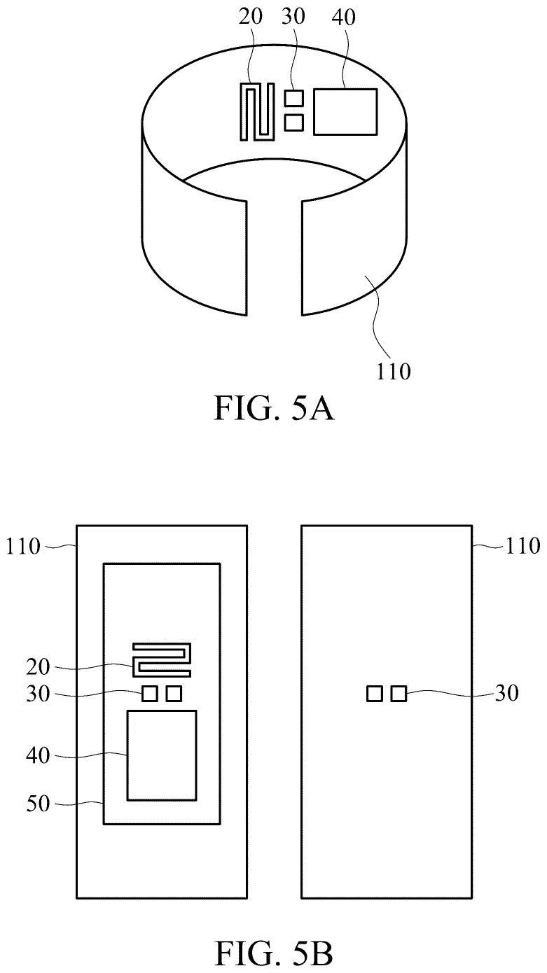

FIG. 5A shows a perspective schematic view of the blood pressure measuring device, according to the fourth embodiment of the present disclosure. FIG. 5B shows the internal configuration and appearance of the first wearable device 110, according to the fourth embodiment of the present disclosure.

In the fourth embodiment, the blood pressure measuring device only includes a single wearable device 110. On the wearable device 110, a BCG sensor 20, a PPG sensor 30, and a power supply element 40 are included. As shown in FIG. 5B, the BCG sensor 20, the PPG sensor 30, and the power supply element 40 are disposed on the flexible printed circuit board 50 and electrically connected to the flexible printed circuit board 50. The flexible printed circuit board 50 is embedded inside the wearable device 110. When viewed from outside, as shown on the right side of FIG. 5B, only the PPG sensor 30 is exposed from the wearable device 110 to be in contact with the human body B. As mentioned above, the BCG sensor 20 only needs to sense vibrations. Thus, it does not need to be in direct contact with the skin of human body B.

In the fourth embodiment, the time difference ΔT_{J-P} between the J-peak of the BCG signal and the P-peak of the PPG signal may be measured. The wearer's blood pressure can be estimated from the time difference ΔT_{J-P}. The estimation method will be described in details below.

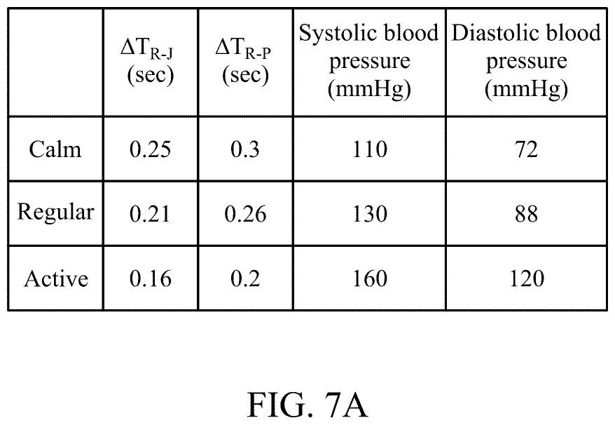

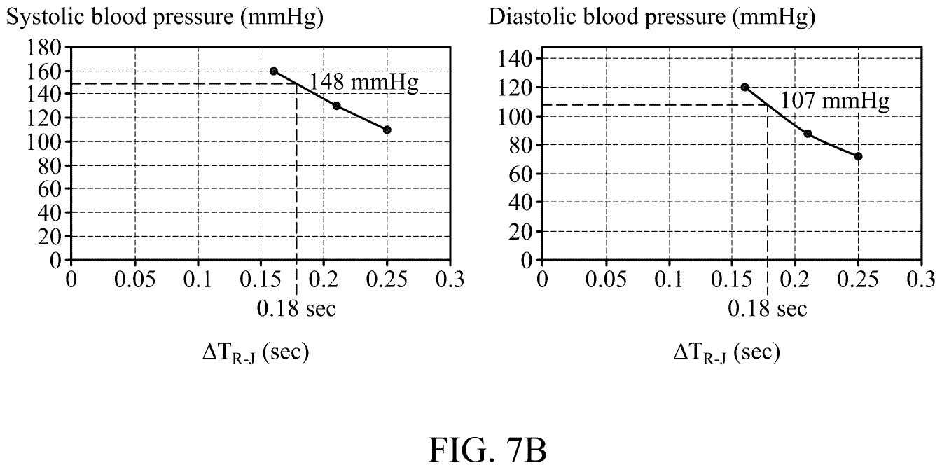

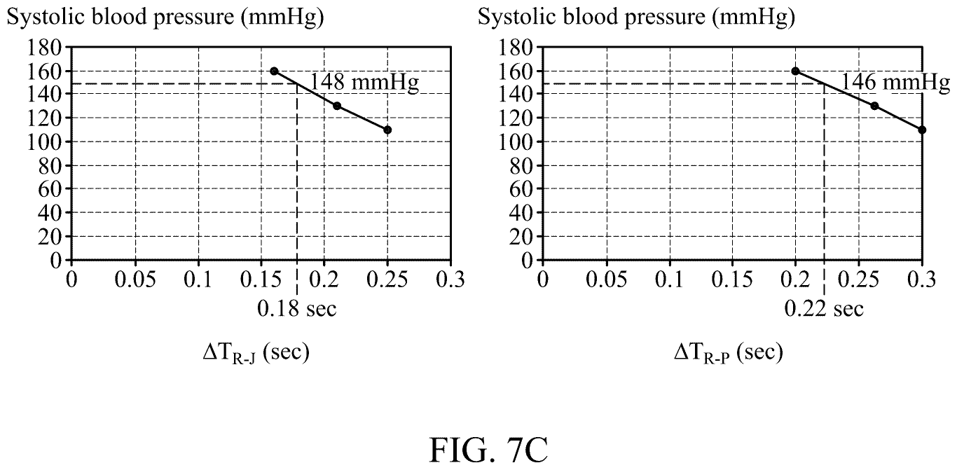

Next, the method for estimating blood pressure will be described in reference to FIGS. 6A, 6B, 7A, 7B, and 7C.

FIGS. 6A and 6B show waveforms of the ECG signal, the BCG signal, and the PPG signal, according to some embodiments of the present disclosure. FIG. 7A shows the correspondence between the time differences and blood pressures, according to some embodiments of the present disclosure. FIG. 7B shows the calibration curve formed based on the correspondence in FIG. 7A, according to some embodiments of the present disclosure. FIG. 7C shows the calibration curve formed based on the correspondence in FIG. 7A, according to some embodiments of the present disclosure.

In the method of estimating blood pressures based on the present disclosure, it is necessary to compare and calibrate using a conventional sphygmomanometer (blood pressure monitor) first. In detail, the blood pressure measured by the conventional sphygmomanometer and the pulse transmission time of the corresponding ECG signal, BCG signal, and PPG signal (either two or all three) are recorded under three states of blood pressure (low blood pressure, normal blood pressure, and high blood pressure). Using the calibration data for the three states (see FIG. 7A), a three-point calibration curve or regression curve (see FIG. 7B) is established. Then, the corresponding blood pressure can be estimated or calculated by measuring any of time difference ΔT_{R-J}, time difference ΔT_{R-P}, or time difference ΔT_{J-P}.

For example, the “Calm” state in FIG. 7A corresponds to a state in which the blood pressure is low; the “Regular” state corresponds to a state in which the blood pressure is normal; and the “Active” state corresponds to a state in which the blood pressure is high. The “Systolic blood pressure” and the “Diastolic blood pressure” are data measured by the conventional sphygmomanometer. The “ΔT_{R-J}” is the calculated average time difference between the R-peak of the ECG signal and the J-peak of the BCG signal. The “ΔT_{R-P}” is the calculated average time difference between the R-peak of the ECG signal and the P-peak of the PPG signal.

The calibration only needs to be performed once. That is, the calibration is completed before the blood pressure measuring device of the present disclosure is activated. Conventional sphygmomanometers are not required for subsequent use of the blood pressure measuring device.

FIG. 7B shows the estimation process using the device of the second embodiment. As shown in FIG. 7B, using the data in FIG. 7A, a calibration curve may be formed using the “ΔT_{R-J}” and the “Systolic blood pressure”, and another calibration curve may be formed using the “ΔT_{R-J}” and the “Diastolic blood pressure”. When the user measures the time difference ΔT_{R-J} of 0.18 seconds using a device such as the one in the second embodiment, the user's systolic blood pressure at that time can be calculated as 148 mmHg and diastolic blood pressure as 107 mmHg by using the two calibration curves.

FIG. 7C shows the estimation process using the device of the third embodiment. As shown in FIG. 7C, using the data in FIG. 7A, a calibration curve may be formed using the “ΔT_{R-J}” and the “Systolic blood pressure”, and another calibration curve may be formed using the “ΔT_{R-P}” and the “Systolic blood pressure”. When the user measures a time difference ΔT_{R-J} of 0.18 seconds and a time difference ΔT_{R-P} of 0.22 seconds using a device such as the one in the third embodiment, two different systolic blood pressures can be calculated by using the two calibration curves. In this case, the average of the two systolic blood pressures can be used as the final output systolic pressure (147 mmHg), or the weighted average of the two can be used as the final output systolic pressure.

Although FIG. 7C only shows the estimation process of “systolic pressure”, the “diastolic pressure” can also be calculated using the same principle. Additionally, although FIG. 7A only shows “ΔT_{R-J}” and “ΔT_{R-P}”, the time difference ΔT_{J-P} measured by the fourth embodiment can also be calculated using the same principle.

In addition, the above estimation process may be performed by a calculating element or processing element of the blood pressure measuring device. The calculating element calculates at least one of the time difference ΔT_{R-J}, the time difference ΔT_{R-P}, or the time difference ΔT_{J-P}, and estimate a blood pressure accordingly. Although the presently disclosed drawings do not illustrate the calculating element, the calculating element may, for example, wirelessly obtain signals from each of the first wearable device 110 and the second wearable device 210, and then perform the calculation. The calculating element may be any suitable calculating element in the technical field, without limitation to the present disclosure.

In summary, the blood pressure measuring device according to the embodiments of the present disclosure does not require an inflatable cuff and does not require to press the action of the pressing sensors to measure blood pressure, just using a wearable device to calculate the blood pressure, which effectively reduces the size of the blood pressure measuring device and improves convenience. The blood pressure measuring device of the present disclosure can calculate any two of the ECG signal, the BCG signal, and the PPG signal to estimate blood pressures from differences in arterial pressure transmission times. It can obtain signals even during sleep and exercise to monitor heartbeat and blood pressure conditions at any time. In addition, the recorded signals can also be used to determine other symptoms and further build a database for monitoring health conditions.

Although embodiments of the present disclosure and their advantages have been described in detail, it should be understood that various changes, substitutions and alterations can be made herein without departing from the spirit and scope of the disclosure as defined by the appended claims. Moreover, the scope of the present application is not intended to be limited to the particular embodiments of the process, machine, manufacture, and composition of matter, means, methods and steps described in the specification. As one of ordinary skill in the art will readily appreciate from the disclosure of the present disclosure, processes, machines, manufacture, compositions of matter, means, methods, or steps, presently existing or later to be developed, that perform substantially the same function or achieve substantially the same result as the corresponding embodiments described herein may be utilized according to the present disclosure. Accordingly, the appended claims are intended to include within their scope of such processes, machines, manufacture, and compositions of matter, means, methods, or steps. In addition, each claim constitutes a separate embodiment, and the combination of various claims and embodiments are within the scope of the disclosure.

What is claimed is:

1. A blood pressure measuring device, comprising:
a first measuring assembly that includes a plurality of sensing elements, which are at least partially in contact with human skin;
a second measuring assembly that includes a sensing element, which is in contact with human skin;
a first wearable device, wherein the first measuring assembly is installed on the first wearable device; and
a second wearable device, wherein the second measuring assembly is installed on the second wearable device.

2. The blood pressure measuring device as claimed in claim 1, wherein the sensing element of the second measuring assembly is an ECG sensor, and the sensing elements of the first measuring assembly include an ECG sensor;
wherein an ECG signal is obtained from the ECG sensor of the first measuring assembly and the ECG sensor of the second measuring assembly.

3. The blood pressure measuring device as claimed in claim 2, wherein the sensing elements of the first measuring assembly further include a PPG sensor, and a PPG signal is obtained from the PPG sensor.

4. The blood pressure measuring device as claimed in claim 3, wherein the sensing elements of the first measuring assembly are disposed on a flexible printed circuit board and electrically connected to the flexible printed circuit board that is embedded inside the first wearable device.

5. The blood pressure measuring device as claimed in claim 4, wherein the ECG sensor and the PPG sensor of the first measuring assembly are exposed from the first wearable device to be in contact with the human skin.

6. The blood pressure measuring device as claimed in claim 3, further comprising a calculating element that calculates a time difference between the ECG signal and the PPG signal, and estimates a blood pressure from the time difference.

7. The blood pressure measuring device as claimed in claim 2, wherein the sensing elements of the first measuring assembly further include a BCG sensor, and a BCG signal is obtained from the BCG sensor.

8. The blood pressure measuring device as claimed in claim 7, wherein the sensing elements of the first measuring assembly are disposed on a flexible printed circuit board and electrically connected to the flexible printed circuit board that is embedded inside the first wearable device.

9. The blood pressure measuring device as claimed in claim 8, wherein the ECG sensor of the first measuring assembly is exposed from the first wearable device to be in contact with the human skin.

10. The blood pressure measuring device as claimed in claim 7, further comprising a calculating element that calculates a time difference between the ECG signal and the BCG signal, and estimates a blood pressure from the time difference.

11. The blood pressure measuring device as claimed in claim 2, wherein the sensing elements of the first measuring assembly further include a PPG sensor and a BCG sensor, and a PPG signal is obtained from the PPG sensor, while a BCG signal is obtained from the BCG sensor.

12. The blood pressure measuring device as claimed in claim 11, wherein the sensing elements of the first measuring assembly are disposed on a flexible printed circuit board and electrically connected to the flexible printed circuit board that is embedded inside the first wearable device.

13. The blood pressure measuring device as claimed in claim 12, wherein the ECG sensor and the PPG sensor of the first measuring assembly are exposed from the first wearable device to be in contact with the human skin.

14. The blood pressure measuring device as claimed in claim 11, further comprising a calculating element that calculates a time difference between any two of the following signals: the ECG signal, the PPG signal, and the BCG signal, and estimates a blood pressure from the time difference.

15. The blood pressure measuring device as claimed in claim 1, wherein the first wearable device and the second wearable device are respectively worn on two hands of the human body.

16. A blood pressure measuring device, comprising:
a measuring assembly that includes a plurality of sensing elements, which are at least partially in contact with human skin;
a wearable device, wherein the measuring assembly is installed on the wearable device; and
a calculating element;
wherein the blood pressure measuring device does not include an inflatable cuff;
wherein the sensing elements of the measuring assembly include a PPG sensor and a BCG sensor, and a PPG signal is obtained from the PPG sensor, while a BCG signal is obtained from the BCG sensor;
wherein the calculating element calculates a time difference between the PPG signal and the BCG signal, and estimates a blood pressure from the time difference.

17. The blood pressure measuring device as claimed in claim 16, wherein the sensing elements of the measuring assembly are disposed on a flexible printed circuit board and electrically connected to the flexible printed circuit board that is embedded inside the wearable device.

18. The blood pressure measuring device as claimed in claim 17, wherein the PPG sensor of the measuring assembly is exposed from the wearable device to be in contact with the human skin.
